(19) United States
(12) Patent Application Publication
(10) Pub. No.: US 2003/0229280 A1
(43) Pub. Date: Dec. 11, 2003

(54) Contrast Medium for Use in Imaging Methods

(75) Inventors: Wolfgang Greb, Dusseldorf (DE); Helmut Blum, Dusseldorf (DE); Marcel Roth, Dusseldorf (DE)

Correspondence Address: GUDRUN E. HUCKETT, LONSSTR. 53, WUPPERTAL, 42289, DE

(73) Assignee: EUCRO EUROPEAN CONTRACT RESEARCH GMBH AND CO. KG, Neuss (DE)

(21) Appl. No.: 10/249,953

(22) Filed: May 22, 2003

(30) Foreign Application Priority Data
May 22, 2002 (DE) 102 22 481.1

(51) Int. Cl. A61B 6/00

(52) U.S. Cl. 600/431

(57) ABSTRACT

An imaging auxiliary for medical imaging methods contains stabilizer-free superparamagnetic particles such as metal oxides and metals. Preferred are Î³—Fe₂O₃, Fe₃O₄, MnFe₂O₄, NiFe₂O₄, and CoFe₂O₄ and their mixtures. The auxiliary can additionally contain biologically and pharmacologically active substances such as proteins, antibodies, peptides, or oligonucleotides. The the superparamagnetic particles have a particle size of 1 nm to 500 nm. A tissue-specific substance in the form of diphosphonic acids and physiologically innocuous salts of diphosphonic acids can be added.

### BACKGROUND OF INVENTION

The present invention relates to the use of superparamagnetic particles as auxiliaries in imaging methods as well as a contrast agent or medium for use in medical diagnostics, in particular, in imaging methods.

Imaging methods, for example, x-ray examinations, MR tomography, scintigraphy etc., represent important resources in medical diagnostics for imaging morphological and metabolic processes in the area of the skeletal systems as well as pathological changes in the area of the soft tissue. When employing these methods, the use of so-called contrast media or agents is generally required. They effect in the body parts to be examined a sharp delimitation between healthy tissue and pathological changes.

Contrast agents in MR tomography are in the form of magnetic particles, for example, superparamagnetic iron oxide particles. A review of such contrast agents is provided in Eur. Radiol. (2001) 11, 2319-2331. The field of application of the described contrast agents comprises essentially the imaging of inner organs such as liver, spleen, and so on.

In the European patent document EP 0 689 430 B1, superparamagnetic particles are disclosed which are comprised of single-domain particles and stabilizer substances, wherein the single-domain particles of iron oxide, mixed iron oxide or iron having a particle size in the range between 3 and 20 nm are aggregated to stable, decomposable aggregates which have a defined behavior in the magnetic field. The particle size of the aggregates is in the range between 10 and 1000 nm. They have a mono-molecular layer on their surface which is comprised of stabilizer substances of the group of: poly alkylene glycols, containing phosphate, diphosphate, carboxylate, polyphosphate, thiophosphate, phosphonate, thiophosphonate, sulfate, sulfonate, mercapto, silane triol, trialkoxy silane groups; carbohydrates; or phosphate group-containing nucleotides, their oligomers or their polymers which may have further bonding sites. Important in the case of the disclosed superparamagnetic particles is that they first aggregate and, subsequently, the stabilizer substances are bonded to the aggregate surface so that the properties of the aggregates change. A possible field of use of the described superparamagnetic particles is the use as a contrast agent for the nuclear spin diagnostics.

In the European patent application EP 0 248 549 A2, liquid magnetic compositions are disclosed which contain a physiologically tolerated dispersion of finely dispersed superparamagnetic particles in water and a sufficient amount of reactive stabilizer substances for the stabilization and for the chemical bonding of diagnostic and pharmacologically active substances. The stabilizer substances are chemically bonded to the surface of the superparamagnetic particles by phosphate, phosphonate or carboxylic groups. These liquid magnetic compositions are used, inter alia, in NMR diagnostics.

The imaging of bones and the like generally is performed by x-ray examination or scintigraphy. By means of scintigraphy, a two-dimensional, intensity-proportional representation of the selective distribution of a Î³-ray emitter in a living organ is obtained. While the conventionally employed contrast agents have disadvantages and side effects such as not being tolerated by the patient, the contrast agents used in scintigraphy are in the form of radio nucleotides which exhibit the long-term side effects of radioactive substances.

The employed contrast agents are administered orally, by infusion or injection. The administered agent is distributed in the blood stream or is distributed by the blood circulation within the body and accumulates in organs. For examining the gastrointestinal tract, the administration of the contrast agent is generally performed orally.

A targeted accumulation of the superparamagnetic particles, for example, in the bone, is not possible with the agents known in the prior art.

Only when these particles are charged with specific tissue-bonding substances as targeting aids, they are bonded organ-specifically and are particularly suitable for imaging purposes.

For the treatment and imaging of bones, bone rebuilding processes, and lesions as well as bone metastases of different genesis, bisphosphonates are used because of their special bonding capability on apatite, the inorganic main component of bone tissue. State of the art in this connection is, for example, technetium-99 bone scintigraphy by means of suitable bisphosphonates which are mostly parenterally administered/injected. For the treatment of bone diseases, they are also administered orally.

The bisphosphonate, which accumulates within the bone apatite, accordingly fixates on the bone the bonded atoms or molecules (drug targeting) which, in turn, can locally develop their activity. In the case of contrast agent application, the iron particles are transported to the bone/skeleton and enable thereat the imaging of bone structure or cavities of the bone marrow (negative imaging). The enrichment on the apatite of the healthy bone and on the apatite of the bone rebuilding zones (for example, growth issues, rheumatoid inflammation processes, bone decomposition or bone tumor events) varies depending on the employed bisphosphonate. The contrast agent can be selected accordingly. SUMMARY OF INVENTION

The present invention has therefore the object to provide an auxiliary for the use in imaging methods that can be enriched in a targeted way in the organ or body part to be examined, wherein the disadvantages or side effects of radiation exposure, as they would occur by radioactive substances or x-rays, are reduced and, if possible, entirely eliminated.

The subject matter of the present invention is therefore the use of stabilizer-free superparamagnetic particles as an auxiliary in imaging methods.

The term auxiliaries in the context of the present invention means that the superparamagnetic particles according to the invention are used as contrast agents or for sensibilization of the physical parameters to be measured, such as nuclear magnetic resonance. For example, the superparamagnetic particles can be employed in x-ray methods as contrast agents, wherein advantage is taken of the effect that the superparamagnetic particles absorb x-rays more weakly or more strongly than the neighboring body tissues/bones so that the representation of the body structures as an image is possible. In the case of nuclear magnetic resonance methods, the superparamagnetic particles according to the invention can effect a contrast enhancement in that they excite/sensibilize hydrogen bonds in their environment, which leads to a stronger measured signal in nuclear magnetic resonance methods.

A further subject matter is a contrast agent or contrast auxiliary for the use in imaging methods, containing stabilizer-free superparamagnetic particles.

The contrast agent or contrast auxiliary (in the following, the expression contrast agent will be used exclusively) is preferably used in nuclear magnetic resonance spectroscopy or tomography and in x-ray imaging.

The employed superparamagnetic particles according to the invention serve in imaging processes as auxiliaries; preferably, they are used as contrast agents or for contrast enhancement in nuclear magnetic resonance spectroscopy or tomography and in x-ray imaging.

In one possible embodiment of the present invention, in addition to the superparamagnetic particles, biological and pharmacological active substances, such as chemical substances, proteins, including antibodies, peptides, oligo nucleotides, are used additionally. In this embodiment, the active substances are transported together with the superparamagnetic particles in a targeted way to the action site. At the action site, the active substances react with special biological agents such as receptors, cells (macrophages), tissue (for example, spleen). They bond actively on such proteins or are actively taken up by cells or tissue. The same holds true also for active substance derivatives, conglomerates, or transport systems with corresponding active groups. According to this embodiment, the active substance transports and bonds the superparamagnetic particles to certain structures in the body where it acts itself as a contrast agent in imaging processes or as an auxiliary in these methods at the local level and makes the targeted structures “visible”.

The superparamagnetic particles contained according to the invention are selected preferably from the superparamagnetic substances suitable for the respective application, such as metal oxides and/or metals, wherein Î³—Fe₂O₃, Fe₃O₄, MnFe₂O₄, NiFe₂O₄, CoFe₂O₄ and any mixtures thereof are especially preferred for in-vivo use because of their physiological compatibility. Fe₃O₄ (magnetite) is particularly preferred. As suitable metals, Fe, Co, Ni as well as their alloys, optionally also with other metals, can be named.

The superparamagnetic particles can be used without the stabilizers described in the prior art, i.e., they are stabilizer-free. The term stabilizer-free means in the present invention that the magnetic particles are charged with active substances, or enveloped by them, without the addition of additives such as emulsifiers or surface coatings as described in the prior art. Also, a treatment of particles charged with active substances is not required and preferably precluded. In the most simple embodiment of the present invention, the system according to the invention is comprised of magnetic particles and active substance.

When the superparamagnetic particles are used in combination with other substances, the latter are usually positioned partially on the surface of the magnetic particles. This means that the additional substances are applied directly onto the surface of the particles. The particles can also be enveloped by the additional substances, which is the case, for example, when the additional substances as a result of their structure, shape or size surround the magnetic particles but are not directly applied to the surface. An envelope is present, for example, when cells, cell cultures or cell components are used as the additional substances, and the magnetic particles are positioned in the interior of the cells, cell cultures or cell components, or when the additional substances, as a result of their molecule size, have a structure of a clew in whose interior the magnetic particles are located.

The superparamagnetic particles used according to the invention have generally preferably a particle size of 1 to 500 nm, preferably of 1-50, in particular, of 10-20 nm, wherein in this connection the individual discrete crystallites are meant. It is also possible that agglomerates are present whose particle size is below 5 Î¼ m, in particular, above 50 nm and below 1,000 nm.

The aforementioned particle size is suitable for any form of administration. However, it was found to be advantageous in connection with oral administration and in connection with slow infusions when the particle size of the superparamagnetic particles is between 50 and 500 nm, in particular, between 200 and 500 nm. Orally administered contrast agents having a particle size in this range are particularly suitable for imaging of the gastrointestinal tract and other hollow organs such as, for example, the bladder, vagina, sinus cavities or cysts as well as blood vessels and open wounds.

When the contrast agent according to the invention is to be administered by infusion or injection, a particle size of the superparamagnetic particles between 1 and 200 nm, in particular, between 1 and 50 nm, was found to be particularly suitable.

For imaging bones or changes on bones, superparamagnetic particles are preferably used in where particle size of the superparamagnetic particles is between 10 and 20 nm.

The volume-weighted average crystallite size can be determined by x-ray diffraction methods, in particular, by means of a Scherrer analysis. This method, for example, is described in C. E. Krill, R. Birringer: “Measuring average grain sizes in nanocrystalline materials”, Phil. Mag. A 77, p. 621 (1998). According to this method, the volume-weighted average crystallite size D can be determined by the efollowing quation:

D=KÎ>>/Î² cos Î,

In this equation, Î>> is the wavelength of the employed x-ray radiation, Î² is the full width at half the height of the reflex on the diffraction position 2Î, K is a constant of the magnitude 1 whose exact value depends on the crystal shape. This indeterminate value of K can be avoided in that the line widening is determined as an integral width Î²ᵢ wherein Ã [symbol] is defined as a surface area underneath the x-ray diffraction reflex divided by its maximum intensity

I₀.

$${β}_{i} = 1 / {I}_{0} {\int }_{2 {θ}_{1}}^{2 {θ}_{2}} I ( 2 θ ) ⅆ ( 2 θ )$$

In this connection, the values 2Î,₁ and 2Î, ₂ are the minimum and maximum angle position of the Bragg reflex on the 2Î, axis. I(2Î,) is the measured intensity of the reflex as a function of 2Î, When employing this equation, the equation for determining the volume-weighted average crystallites size D is as follows:

D=Î>>/Î²ᵢ cos Î,

It was found that the diphosphonic acids and their salts contained according to the invention as a tissue-specific substance have the capability to bond in a targeted way to the structural tissue because of the active molecule groups contained in the molecules so that an especially excellent enrichment of the contrast agent in the organ to be examined takes place.

In a preferred embodiment, the superparamagnetic particles are used in combination with a tissue-specific substance, selected from diphosphonic acids and their physiologically innocuous salts. The diphosphonic acids and their physiological innocuous salts are characterized by their excellent bonding properties on body tissue, in particular, calcified tissue (calcification) and with respect to bones. In addition to the bones/skeleton as a positive target, the corresponding cavities are also represented, for example, bone marrow. The combination of superparamagnetic particles and diphosphonic acid or its physiologically innocuous salts is particularly advantageous for imaging bones, i.e., the skeleton, and, in particular, of fine structures or rebuilding zones such as metastases. The diphosphonic acid and its physiologically innocuous salts accumulate in the rebuilding zones, such as metastases, so that, in the case of a combination comprising superparamagnetic particles and diphosphonic acid (salt), the superparamagnetic particles are enriched also and show the structural change in the x-ray image. Because of the insufficient imaging properties of fine structures and rebuilding zones, currently a radioactive scintigraphy by use of technetium compounds is regularly carried out when there is a suspicion with regard to metastases in the bone structure.

Geminal diphosphonic acids as well as geminal bisphosponic carboxylic acids and their physiologically innocuous salts with the general formula I were found to be particularly suitable diphosphonic acids:

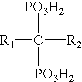

wherein:—R¹ is COOH, a linear or branched alkyl group with 1 to 10 carbon atoms, which can be optionally substituted by substituents such as amino groups, NBmonoalkylamino groups or N-dialkylamino groups, wherein the alkyl groups can contain 1 to 5 C atoms and/or SH groups, or a substituted or unsubstituted carbocyclic or heterocyclic aryl/cycloalkane group, which can also form a condensed ring system with up to three rings, which can optionally contain one or several hetero atoms, especially preferred are N atoms as hetero atoms, and as substituents can contain branched or unbranched alkyl groups with 1 to 6 C atoms, free or mono-alkylated or di-alkylated amino groups with 1 to 6 C atoms, or halogen atoms; and—R₂ is OH, COOH, a halogen atom, preferably Cl, H or NH₂.

Alkali metal salts, alkaline earth metal salts, ammonium salts and/or ethanolamine salts can be mentioned as examples of suitable salts of the compounds of the formula I.

Such substances are suitable in particular for the treatment of osteoporotic diseases, wherein the following compounds are particularly preferred.

1-methyl-1-hydroxy-1,1-diphosphonic acid (MDP).

1,1-diphosphono propane-2,3-dicarboxylic acid (DPD).

3-(methyl pentyl amino)-1-hydroxy propane-1,1-diphosphonic acid (ibandronic acid).

1-hydroxyethane-1,1-diphosphonic acid (editronic acid; HEDP).

dichloromethane diphosphonic acid (clodronic acid).

3-amino-1-hydroxypropane-1,1-diphosphonic acid (pamidronic acid).

4-amino-1-hydroxybutane-1,1-diphosphonic acid (alendronic acid).

2-(3-pyridine)-1-hydroxyethane-1,1-diphosphonic acid (risedronic acid).

4-chlorophenyl thio methane-1,1-diphosphonic acid (tiludronic acid).

pyrimidinyl-1-hydroxyethane-1,1-diphosphonic acid (zoledronic acid).

cycloheptyl amino methane-1,1-diphosphonic acid (cimadronic acid).

6-amino-1-hydroxyhexane-1,1-diphosphonic acid (neridronic acid).

3-(N,N-dimethylamino)-1-hydroxypropane-1,1-diphosphonic acid (olpadronic acid).

3-pyrrol-1-hydroxypropane-1,1-diphosphonic acid.

and/or 2-pyrimidazole-1-hydroxyethane-1,1-diphosphonic acid (minodronic acid).

azacycloheptane-2,2-diphosphonic acid as well as their physiological tolerated salts.

The diphosphonic acids as a tissue-specific substance can be applied in any suitable way onto the superparamagnetic particles or bonded thereto. Conventionally, the diphosphonic acids are located on the surface of the superparamagnetic particles and/or, when they are present as agglomerates, also in their cavities. The diphosphonic acids can also be bonded to the particles by covalent and/or ionic bonds, optionally by means of spacer groups, or by means of Van-der-Waal's forces.

The contrast agent according to the invention contains superparamagnetic particles as well as a tissue-specific substance, wherein the tissue-specific substance preferably is adsorbed on the superparamagnetic particles. In one possible embodiment, the superparamagnetic particles and the tissue-specific substances can be mixed together as solid materials. However, it was found to be particularly advantageous when the tissue-specific substance is already present when forming the magnetic particles, for example, when the superparamagnetic particles are produced by a size-controlled precipitation in aqueous medium by means of alkaline substances or by reduction of metal cations. As a result of the large particle surfaces produced in situ, an optimal adsorption of the tissue-specific substances on the surface of the particles by means of reactive groups such as OH, SH, hydroxide, amino, carboxyl, ether, sulfo, phosphonic acid groups and so on. It is also possible to apply the tissue-specific substance subsequently onto the precipitated superparamagnetic particles, for example, by suspension of the un-coated (unmodified) superparamagnetic particles in a liquid phase, preferably water, containing the tissue-specific substance or a substance mixture.

In one possible embodiment of the present invention, the active substances can also be bonded by so-called spacer groups on the magnetic particles. Spacers are short organic molecule chains which are used for immobilization of molecules on carriers, wherein the spacer molecules do not represent a coating. Spacers can be used, for example, when the active substances have no polar groups or ionic groups. The spacer molecules can improve bonding between the magnetic particles and the active substances. They have preferably one or several polar groups. As examples, reference can be had to the already mentioned groups. In particular, when cationic active substances are used, spacers with two polar groups such as amino carboxylic acids, diamines, betains, dicarboxylic acids, amino phosphonates etc. have been found to be suitable.

In a further possible embodiment, so-called agglomerates of magnetic particles are used which are comprised of agglomerates of nanoparticles, i.e., of crystallites having a particle size of less than 100 nm. These agglomerates can be comprised of individual crystallites which are either reversibly agglomerated at their contact surface or irreversibly agglomerated by means of covalescence, i.e., by growing together past the boundary of the grain. An advantage of agglomerates resides in that they have an outer as well as an inner surface, i.e., have cavities, so that the tissue-specific substance can be bonded in the interior and on the exterior. Agglomerates can be obtained, for example, in that the magnetic particles are precipitated in the absence of an active substance, by drying or freeze-drying of particles free of active substance or charged with tissue-specific substance with subsequent re-dispersing, agglomerate formation, which can be controlled by the conditions during synthesis such as temperature increase, adjustment of the pH value, high electrolyte contents, or by a suitable after treatment of the precipitated particles at temperatures above 100 degrees C.

The agents according to the invention are used as conventional pharmaceutical administration forms, i.e., parenterally, intravenously, by inhalation, orally, instillation in body cavities, even intraoperatively.

The contrast agent according to the invention, as mentioned above, can be applied orally, by infusion or injection. For these forms of administration, the contrast agent according to the invention is preferably converted into a suitable pharmaceutical composition, wherein particularly suspensions, emulsions, or liposome systems are to be mentioned. For the oral administration, tablets or capsules can be mentioned additionally.

The contrast agent according to the invention is used for improved imaging in medical diagnostics of organs and body parts to be examined. Particularly suitable imaging methods are MR tomography and other nuclear spin methods for macroscopic and microscopic imaging.

A further subject matter of the present invention is accordingly the use of the afore described contrast agent in medical diagnostics, in particular, in MR tomography for imaging the bony skeleton and lesions on bones.

### DETAILED DESCRIPTION

### EXAMPLE

### Preparative Examples

1. In 40 g of deionized water, 6.48 g FeCl₃ were dissolved. Also, 3.97 g FeCl₂ C4H₂O was dissolved in a mixture of 8 ml deionized water and 2 ml 37% hydrochloric acid. The two mixtures were combined shortly before use of the solutions in the precipitation process.

2. In a beaker, 400 ml deionized was stirred with 10 g NaOH and 0.2 g 1-methyl-1-hydroxy-1,1-diphosphonic acid (MDP). After cooling, the hydrochloric acid iron solution prepared in 1 was added with intense agitation. By means of a magnetic field, the formed black precipitate was sedimented and the solution above was decanted. Subsequently, water was added several times to the precipitated material and decanted in order to remove foreign ions. Subsequently, 0.5 g MDP and 100 ml water were added. After stirring for an hour at 40 degrees C., the mixture was stirred for 12 hours at room temperature. Portions that were not suspended were separated by centrifugation (5,000-11,000 revolutions/minute). In this way, a magnetic liquid was obtained which was concentrated to the point of obtaining the desired solids contents in a rotary evaporator.

3. Example 2 was repeated wherein, instead of 1-methyl-1-hydroxy-1,1-diphosphonic acid, 1,1-diphosphono propane-2,3-dicarboxylic acid (DPD) was used.

What is claimed is:

1. An imaging auxiliary for medical imaging methods, comprising:
stabilizer-free superparamagnetic particles, selected from the group consisting of metal oxides and metals.

2. The imaging auxiliary according to claim 1, wherein the metal oxides are selected from the group consisting of Î³Fe₂O₃, Fe₃O₄, MnFe₂O₄, NiFe₂O₄, and CoFe₂O₄.

3. The imaging auxiliary according to claim 2, comprising a mixture of at least some of the metal oxides.

4. The imaging auxiliary according to claim, further comprising biologically and pharmacologically active substances.

5. The imaging auxiliary according to claim 4, wherein the biologically and pharmacologically active substances are selected from the group consisting of chemical substances, proteins, antibodies, peptides, or oligonucleotides.

6. The imaging auxiliary according to claim 1, wherein the superparamagnetic particles have a particle size of 1 nm to 500 nm.

7. The imaging auxiliary according to claim 6, wherein the particle size of the superparamagnetic particles is 1 nm to 50 nm.

8. The imaging auxiliary according to claim 7, wherein the particle size is 10 nm to 20 nm.

9. The imaging auxiliary according to claim 1, further comprising a tissue-specific substance selected from the group consisting of diphosphonic acids and physiologically innocuous salts of said diphosphonic acids.

10. The imaging auxiliary according to claim 9, wherein said diphosphonic acids are geminal diphosphonic acids of the formula 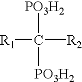
wherein:
R₁ is COOH, a linear or branched alkyl group with 1 to 10 carbon atoms, which can be optionally substituted by substituents such as amino groups, N-monoalkylamino groups or N-dialkylamino groups, wherein the alkyl groups can contain 1 to 5 C atoms and/or SH groups, or a substituted or unsubstituted carbocyclic or heterocyclic aryl/cycloalkane group, which can also form a condensed ring system with up to three rings, which can optionally contain one or several hetero atoms, especially preferred are N atoms as hetero atoms, and as substituents can contain branched or unbranched alkyl groups with 1 to 6 C atoms, free or mono-alkylated or di-alkylated amino groups with 1 to 6 C atoms, or halogen atoms; and
R₂ is OH, COOH, a halogen atom, Cl, H or NH₂.

11. The imaging auxiliary according to claim 10, wherein said diphosphonic acids are selected from the group consisting of 1-methyl-1-hydroxy-1,1-diphosphonic acid (MDP), 1,1-diphosphonopropane-2,3-dicarboxylic acid (HPD), 3-(methyl pentyl amino)-1-hydroxy propane-1,1-diphosphonic acid (ibandronic acid), 1-hydroxyethane-1,1-diphosphonic acid (editronic acid), dichloromethane diphosphonic acid (clodronic acid), 3-amino-1-hydroxypropane-1,1-diphosphonic acid (pamidronic acid), 4-amino-1-hydroxybutane-1,1-diphosphonic acid (alendronic acid), 2-(3-pyridine)-1-hydroxyethane-1,1-diphosphonic acid (risedronic acid), 4-chlorophenyl thio methane-1,1-diphosphonic acid (tiludronic acid), pyrimidinyl-1-hydroxyethane-1,1-diphosphonic acid (zoledronic acid), cycloheptyl amino methane-1,1-diphosphonic acid (cimadronic acid), 6-amino-1-hydroxyhexane-1,1-diphosphonic acid (neridronic acid), 3-(N,N-dimethylamino)-1-hydroxypropane-1,1-diphosphonic acid (olpadronic acid), 3-pyrrol-1-hydroxypropane-1,1-diphosphonic acid and/or 2-pyrimidazole-1-hydroxyethane-1,1-diphosphonic acid (minodronic acid).

12. The imaging auxiliary according to claim 1, in the form of a suspension, an emulsion, or a liposome system.

13. In a method for medical imaging, the improvement which comprises:
administering an effective amount of the imaging auxiliary according to claim 1.

14. The improvement according to claim 13, wherein the effective amount is administered parenterally, intravenously, by inhalation, orally, by instillation in body cavities, or intraoperatively.

15. The improvement according to claim 13, comprising the step of imaging bony skeleton and lesions on bones.

16. In a method for medical imaging by nuclear magnetic resonance spectroscopy or tomography, the improvement which comprises:
enhancing contrast by administering an effective amount of the imaging auxiliary according to claim 1.

17. The improvement according to claim 16, wherein the effective amount is administered parenterally, intravenously, by inhalation, orally, by instillation in body cavities, or intraoperatively.

18. The improvement according to claim 16, comprising the step of imaging bony skeleton and lesions on bones.

19. In a method for x-ray imaging, the improvement which comprises:
enhancing contrast by administering an effective amount of the imaging auxiliary according to claim 1.

20. The improvement according to claim 19, wherein the effective amount is administered parenterally, intravenously, by inhalation, orally, by instillation in body cavities, or intraoperatively.

21. The improvement according to claim 19, comprising the step of imaging bony skeleton and lesions on bones.
